(19) United States
(12) Patent Application Publication
(10) Pub. No.: US 2003/0050687 A1
(43) Pub. Date: Mar. 13, 2003

(54) Biocompatible stents and method of deployment

(76) Inventors: Nathan D. Schwade, Dallas, TX (US); Meital Zilberman, Dallas, TX (US)

Correspondence Address: Sanford E. Warren, JR., GARDERE WYNNE SEWELL LLP, Suite 3000, 1601 Elm Street, Dallas, TX, 75201, US

(21) Appl. No.: 09/898,656

(22) Filed: Jul. 3, 2001

(51) Int. Cl. A61F 2/06

(52) U.S. Cl. 623/1.15

(57) ABSTRACT

The present invention is a structural support stent for use in a body lumen made up of a self-expanding biocompatible material that forms a cylinder having an inner surface and an outer surface. Advantages of the present invention include its self-expanding nature and ease of manufacturing. The present invention also has an ability to deliver drugs in a time-released fashion.

### FIELD OF INVENTION

The present invention relates generally to the field of medical devices and more particularly to methods and devices for use as stents in a body lumen.

### BACKGROUND OF THE INVENTION

Metal and polymeric stents have been successfully used in a variety of applications, but there are some limitations. Most current stents used in the airway require a balloon catheter for deployment, which is counterproductive when using a bronchoscope because the balloon blocks visualization. Therefore, balloon catheter devices require the use of a fluoroscope. Moreover, the current stents are usually titanium and are therefore permanent. The fact that metal stents are permanent poses a problem in children as the child continues to grow because the cross-sectional area of the trachea increases. Eventually the stent itself becomes an obstruction and must be removed by a surgeon. There have been associated problems caused by the removal of the metal stents.

Several polymeric stents for various applications have been reported, such as a tubular poly (L-lactic acid) (PLLA)/polycaprolactone microporous stent for delivering gene transfer vectors to the arterial wall, a spiral PLLA stent, and a tubular poly (DL-lactic acid) and poly (DL lactic-co-glycolic acid) stent, both for urethral applications.

The present inventors have recognized that a bioresorbable stent that allows for endoscopic deployment and which supports, for instance, the neonatal trachea in tracheal malacia until the airway matures, thereafter being totally resorbed, should reduce many of the problems associated with traditional devices.

### SUMMARY OF THE INVENTION

The present invention is a structural support stent for use in a body lumen made up of a self-expanding biocompatible material that forms a cylinder having an inner surface and an outer surface. Advantages of the present invention include its self-expanding nature and ease of manufacturing. The present invention also has the ability to deliver drugs in a time-released fashion.

One embodiment of the present invention is a method of preparing a self-expanding support stent having an inner surface and an outer surface that begins with the forming of a film of a biocompatible material and rolling the film onto a mandrel. The next step is annealing the film while it is on the mandrel. The final step is removing the cured film from the mandrel.

Another embodiment is a method of creating a self-expanding support stent that begins with preparing a solution containing a biocompatible material and one or more drugs, and then casting a film of the biocompatible material and drugs. The next stage is rolling the film onto a mandrel, followed by annealing the film while it is on the mandrel.

Yet another embodiment is a method for deploying a self-expanding structural support stent that begins with reducing the diameter of the stent by coiling and then stabilizing the coiled stent in the coiled position. The next process step is placing the stent in a body lumen, followed by releasing the stabilization of the coiled stent to allow self-expansion.

Another embodiment is a method for creating a gradient of drug across the thickness of a biocompatible film that begins with dissolving a biocompatible material and suspending a finely divided drug in a solvent. A film is formed, using relatively fast drying, from the dissolved biocompatible material and suspended drug while the drug is still suspended. The method next involves rolling the film onto a mandrel and annealing the film while it is on the mandrel. The final step in the method is removing the annealed film from the mandrel.

Another embodiment of the present invention is a method for creating a gradient of drug across the thickness of a biocompatible material that begins by dissolving or suspending a finely divided drug in a solvent and forming a film using relatively slow drying from the dissolved biocompatible material or the suspended drug. Next, the film is rolled onto a mandrel and the film is annealed while it is on the mandrel. Lastly, the annealed film is removed from the mandrel.

The present invention further discloses a method for creating a gradient of a drug across a biocompatible film. This method entails the preparation of a several biocompatible films with different drug contents, binding of these films together to create a multi-layer film, rolling the multi-layer film onto a mandrel, annealing the multi-layer film while it is still on the mandrel, and removing the multi-layered film from the mandrel.

### BRIEF DESCRIPTION OF THE DRAWINGS

FIG. 1 depicts stress-strain curves in tension of H-PLLA films in accordance with the present invention;

FIG. 2 depicts the tensile mechanical properties of H-PLLA films in accordance with the present invention;

FIG. 3 depicts the degree of crystallinity and weight loss of the H-PLLA films in accordance with the present invention;

FIG. 4 depicts stress-strain curves of H-PLLA and L-PLLA films in accordance with the present invention;

FIG. 5 depicts the effect of degradation time on the mechanical properties of H-PLLA, H-PLLA/DM(A), H-PLLA/DM(B) in accordance with the present invention;

FIG. 6 depicts the tensile strength and degradation time of H-PLLA and L-PLLA in accordance with the present invention;

FIG. 7 depicts weight retention and degradation time of H-PLLA and L-PLLA in accordance with the present invention;

FIG. 8 represents various modes of the dilated stent in accordance with the present invention;

FIG. 9 depicts radial elastic deformation in H-PLLA, H-PLLA/DM(A), and H-PLLA/DM(B) in accordance with the present invention; and

FIG. 10 depicts longitudinal fracture in H-PLLA, H-PLLA/DM(A), and H-PLLA/DM(B) in accordance with the present invention.

FIG. 11 depicts a stent with structural supports in accordance with the present invention;

FIG. 12 depicts a stent with ends of different diameter in accordance with the present invention;

FIG. 13 depicts a stent with two differently sized cylinders in accordance with the present invention;

FIG. 14 depicts a stent having two ends of different diameter and a middle section with a smaller diameter than both of the ends in accordance with the present invention;

FIG. 15 depicts a cross-sectional view of a stent having two polymer films in accordance with the present invention;

FIG. 16 depicts a cross-sectional view of an external locking mechanism for the stent in accordance with the present invention;

FIG. 17 depicts a cross-sectional view of an internal locking mechanism for the stent in accordance with the present invention.

### DETAILED DESCRIPTION OF THE INVENTION

Although making and using various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention, and do not delimit the scope of the invention.

Many of the stents used currently for blood vessel and/or airway support have the disadvantage of not being self-expandable. Moreover, when growth occurs, the stent needs to be removed via an invasive procedure. These procedures have resulted in serious complications and even death.

The term “stent” herein means a medical implant in the form of a hollow cylinder, which when implanted into contact with a site in the wall of the lumen to be treated will provide structural support for the body lumen. The structural support is necessary both to prevent collapse of blood vessels, and to provide a framework for tissue growth. The stent disclosed herein is self-expanding, is relatively easy to manufacture, is made from biocompatible material, and can deliver drugs in a time-released fashion. Hence, the stent has a number of advantages over currently used stents, and can have a variety of uses within the body.

Materials

The following materials and methods were used to prepare stents in accordance with the present invention. These materials and methods are merely illustrative and those skilled in the art will appreciate that other biocompatible materials may be used in a manner similar to those described.

High molecular weight Poly(L-lactide) (H-PLLA), RESOMER L21 (i.v.=3.6 dL/g in CHCl₃ at 30° C.), Boehringer Ingelheim, Germany. Low molecular weight Poly(L-lactide) (L-PLLA), L-PLA (i.v.=1.02 dL/g in CHCl₃ at 30° C.), Birmingham Polymers, USA. Poly(DL-lactide) (PDLLA), DL-PLA (i.v.=1.07 dL/g in CHCl₃ at 30° C.), Birmingham Polymers, USA.

Non-inflammatory drugs. Dexamethasone(DM)USP,9α-Fluoro-162-methylprednisolone, SIGMA D-9184. Hydrocortisone, 11,17,21-Trihydroxypregn-4-ene-3,20-dione, CALBIOCHEM 3867. Curcumin, 1,7-bis[4-Hydroxy-3-methoxyphenyl]-1,6-heptadiene-3,5-dione, SIGMA C-1386.

Film Preparation. Polymer films (0.12-0.15 mm thickness) of poly-lactic acid and dexamethasone were prepared as follows:

(a) Components were mixed in chloroform at room temperature until polymer dissolution. A constant (polymer+drug) quantity was chosen, in most situations DM content is 2% wt. In others, contents of 3, 5, and 10% wt are used. For each polymer/drug composition two kinds of solution were prepared, diluted, and concentrated. The solubility of DM in chloroform at room temperature is approximately 1 mg/ml. Dilute solutions were prepared by using a relatively large volume of chloroform. (Concentration range of the polymer: 0.01-0.02 gr/ml with a concentration range of DM: 2×10⁻⁴-10⁻³ gr/ml). Both DM and Poly-lactic acid were totally dissolved. Concentrated solutions were prepared by using a relatively small volume of chloroform. Concentration range of the polymer: 0.05-0.1 gr/ml with a concentration range of DM: 10⁻³-5×10⁻³ gr/ml). In concentrated solutions, the polymer was totally dissolved, while the DM powder is only broken into small particles (aggregates), which yielded an opaque solution.

(b) Solution casting into a petri dish and solvent drying was conducted at room temperature. Two solvent evaporation rates were used: a relatively slow rate of 2-5 ml/hr and a relatively fast rate of 10-20 ml/hr.

(c) Isothermal heat treatment at 90° C. for 1 hour in a vacuum oven.

Morphological Characterization. Films. Polarized light microscopy (LM) was performed using an Olympus BHS compound microscope and a Nikon Diathot inverted light microscope. Transmission electron microscopy (TEM) of ultramicrotomed samples was performed using a Jeol 1200 EX II at an accelerating voltage of 80 kV. High-resolution scanning electron microscopy (HRSEM) of cryogenically fractured surfaces is performed using a Leo Gemini-982, at an accelerating voltage of 1 kV.

Dexamethasone powder. Scanning electron microscopy (SEM) was performed for the drug powders, using a Jeol JSM 840 A at an accelerating voltage of 10 kV. The SEM samples were gold sputtered prior to observation.

Thermal Analysis. Melting temperature (Tₘ), heat of fusion (ΔHₘ) and degree of crystallinity (% C) were determined by differential thermal analysis using an indium calibrated TA Instruments DSC 2010 differential scanning calorimeter (DSC). The measurements were carried out on 10 mg samples under N₂ atmosphere, heating the samples from 30° C. to 250° C. (above their melting points). The analysis was performed with TA Universal Analysis software. The degree of crystallinity, % C, was calculated by the following relationship:

$$\% C = \frac{Δ {H}_{m}}{Δ {H}_{F}} \times 100$$

where ΔHₘ is the measured heat of fusion of the semicrystalline sample and ΔH_{F} is the heat of fusion of the perfect crystal (93.6 J/gr for PLLA).

Mechanical Property Measurements. The mechanical properties of the films were measured at room temperature in unidirectional tension at a rate of 10 mm/min (ASTM D 882-97), using a Universal Testing System machine, MTS Systems Corporation, Eden Prairie, Minn. The tensile strength is defined as the maximum strength in the stress-strain curve; the maximal strain as the breaking strain; the Young's modulus as the slope of the stress-strain curve in the elastic (linear) region.

A chamber was used to measure the radial compression strength of the stents. The chamber permits hydrostatic pressure to be applied to the external surface of the stent. Five samples were tested for each point, for both mechanical and property measurements.

In Vitro Studies. To determine the degradation rate, films were weighed and then floated on sterile water at 37° C. One side of the film was exposed to water and the other side exposed to water-saturated air, to simulate the conditions in the trachea. Every two weeks samples were removed, dried in a vacuum oven, and weighed. The weight loss was calculated as:

$$W e i g h t l o s s ( \% ) = 100 x \frac{{w}_{0} - {w}_{f}}{{w}_{0}}$$

where wₒ and w_{f} are the weights of the dried films before and after water exposure, respectively.

To determine the degradation of tensile mechanical properties, film samples were also floated on sterile water at 37° C. for certain periods of times and then removed, dried and tested as described in the mechanical properties measurements section. In addition, stents formed from films were immersed in sterile water, as before, then studied for the degradation in their radial compression strength.

The Morphology of PLA/Dexamethasone Films. The film structures created from the diluted and concentrated solutions were termed as “A” and “B”, respectively. A comparison of L-PLLA/DM(A), L-PLLA/DM(B), H-PLLA/DM(A) and H-PLLA/DM(B) films containing 2% wt DM and treated at 90° C. with the corresponding neat matrix films, L-PLLA and H-PLLA was made using polarized LM. The melting points of these films and their degree of crystallinity are presented in Table 1.

TABLE 1
Melting Temperature and Degree of Crystallinity of PLLA Based Films.

| | Melting | |
|---|---|---|
| Film | Temperature (° C.) | Degree of Crystallinity (%) |
| L-PLLA | 174 | 53.6 |
| L-PLLA/DM (A) | 173 | 52.2 |
| L-PLLA/DM (B) | 174 | 52.0 |
| H-PLLA | 181 | 41.4 |
| H-PLLA/DM (A) | 181 | 38.2 |
| H-PLLA/DM (B) | 181 | 37.5 |

Large spherulites (50-100 μm) were observed for the L-PLLA film, as can be expected for a highly crystalline polymer (53.6% C). The H-PLLA film is less crystalline than the L-PLLA one (41.4% C) and its spherulites are relatively small (less than 10 μm). The structure and crystallinity differences of these polymers result mainly from the molecular weight difference. While the relatively short L-PLLA chains (i.v.=1.0 dL/gr, corresponding to approximately 130 kD) are likely to crystallize, the long H-PLLA chains (i.v.=3.6 dL/gr, corresponding to approximately 380 kD) are more difficult to crystallize. The melting temperature of the H-PLLA film is higher than that of L-PLLA, probably due to the existence of “larger” i.e., more “perfect” crystals. In order to observe the H-PLLA structure, a different degree of polarization was used.

Large rectangular DM crystals (50-300 μm) were observed on the surface of both PLLA(A) films. These crystals can be observed without a polarizer. In both cases, the structure of the polymer film (below the DM crystals) was similar to that of the neat matrix polymer, indicating that separate crystallization processes occur for PLLA and DM at different drying stages. Each DM crystal is actually composed of smaller crystals, gathered during an advanced crystallization stage. The degree of crystallinity of the A type polymer containing DM PLLA films (52.2% C for L-PLLA/DM(A) and 38.2% C for H-PLLA/DM(A) were very similar to these of the neat PLLA films (53.6% C for L-PLLA and 41.4% C for H-PLLA).

LM observations of B type PLLA/DM films showed a difference in structure, compared to that of the neat matrix polymers. Most of the DM was located within the PLLA film and only a small part was located on the surface. Although a polarizer was used, the polymer characteristic features within the L-PLLA/DM(B) film cannot be readily observed, due to distribution of the drug within the polymer film disturbing the birefringence effects. Since the degree of crystallinity of this film was also high (52.0% C) and similar to that of the neat L-PLLA (53.6% C), the L-PLLA film was also arranged in a spherulitic structure. However, the spherulites of the PLLA within the L-PLLA/DM(B) film were smaller than those of the neat L-PLLA film.

A similar morphology, where most of the DM was dispersed within the polymer matrix, was also observed for the H-PLLA/DM(B) film. In this case, the polymer structure can be observed, due to the relatively high degree of polarization. It appears that the spherulites of the PLLA within the H-PLLA/DM(B) film were similar to those building the neat H-PLLA film. The morphology study of the PLLA/DM(B) films indicates that in order to incorporate the drug in the polymer film, the two components should crystallize in parallel. Interestingly, for both PLLAs the melting temperature was not affected by DM incorporation in the film (Table 1), indicating that the DM does not affect the PLLA crystal's “size” and shape.

Similar morphologies were observed for the corresponding films containing 3, 5, and 10% wt DM within the PLLA matrix. To better understand the effect of the matrix polymer structure on the DM distribution, PDLLA-based films were prepared. The two basic types of solutions, diluted and concentrated, yielded the A and B film types as revealed by polarized LM. The PLLA matrix was amorphous and did not show any typical structure. Hence, the DM mode of dispersion depends mainly on the starting solution and derived parameters (discussed below). The type of poly-lactide affects the polymer morphology, but also has a minor effect on the drug distribution in the polymer. The chemical structure of DM is different than that of PLLA and therefore, for the B type films based on semi-crystalline matrices, most of the DM was located around the PLLA spherulites, in amorphous domains. For the B type films based on semi-crystalline matrices, the DM crystals were located within the amorphous regions of a semi-crystalline polymer, around the spherulites. A finer DM dispersion was obtained within the amorphous PDLLA matrix, due to the absence of crystalline structure features, i.e., the DM was not “directed” to certain domains within the matrix polymer.

Structuring of Solution-Cast PLA/Drug Films. As discussed, two extreme structures were created: (a) A polymer film with large DM crystals located on its surface and (b) A polymer film with small drug crystals and particles located within the bulk. The process of film creation during solvent drying was studied to determine the structures created and to understand how the various processing parameters affect the film morphology. Various points of A and B L-PLLA/DM film formation were observed by inverted LM. The polymer crystallization process could not be observed via inverted LM, and therefore, polarized compound LM was used to complete the study.

The A type PLLA/DM film formation process was as follows:

(a) Nucleation of DM particles on the solution surface. Since the highest rate of solvent evaporation is obtained near the solution/air interface, the primary drug nucleation occurs there. The solution was diluted at this stage.

(b) The concentration of the nuclei increases in parallel to their growth. Hence, many 1-10 μm particles and aggregates were observed. The polymer solution was less diluted in this stage.

(c) The DM particles segregate, due to inter-particle interactions, to form ordered shapes. The polymer solution was relatively viscous, since most of the solvent has already been evaporated.

(d) The DM particles were merged to form large rectangular and hexagonal crystals. A spherulitic structure was observed beneath, within the gel-like solution. PLLA crystallization started during stage (c), at least near the surface where the solvent evaporation rate was relatively high. However, lamellae or very small spherulites cannot be observed via polarized LM, due to lack of birefringence effects. Therefore, the polymer structure was not observed before this stage.

(e) Final drug crystals of characteristic dimensions of 50-300 μm are obtained.

The B type PLLA/DM film formation process was as follows:

(a) Nucleation of DM particles in a very viscous solution and also on its surface. The L-PLLA starts its crystallization.

(b) The DM particles grew, in parallel to the polymer crystallization. Many 1-10 μm particles were observed.

(c) Segregation of DM particles within the viscous medium of the crystallizing polymer. The PLLA spherulites were relatively big and the DM particles migrated to the amorphous domains and around the spherulites, and accumulated there.

(d) A semi-network of DM crystals was created in the amorphous domains of the semi-crystalline PLLA matrix. A higher magnification indeed showed the rounded shape “spherulitic features” of the matrix polymer.

In order to obtain an A-type polymer/drug film, where the drug is located on the surface of the polymer, both components need to be fully dissolved in a common solvent. A relatively low molecular weight drug tends to crystallize before the high molecular weight polymer. Therefore, during a slow evaporation process, drug nucleation occurs on the surface of the solution, where the highest drying rate is obtained. The slow evaporation stage is accompanied by diffusion of DM molecules from the solution to its surface and skin formation. A later polymer core formation occurs in parallel, to further drug particle merging and crystallization on its surface. In contrast, in order to obtain a B-type polymer/drug film, where the drug is distributed within the polymer, a relatively concentrated solution must be used, i.e., the polymer solution contains fine drug particles not totally dissolved. Thus, there is over saturation of drug within the solution. After casting, parallel crystallization of both drug and polymer occurs, due to the relatively rapid drying, deriving from the small solvent quantity. Since the nature of the drug is different than that of the matrix polymer, the drug particles migrate to the amorphous domains of the crystallizing polymer, where they segregate to form a semi-network structure within the matrix polymer. The DM particles are actually “trapped” in the viscous medium. Also, the concentrated solution contains DM particles rather than molecules, which diffuse more slowly. Therefore, a DM core formation is not favored in the B-type polymer/drug film.

To determine the effect of solvent evaporation rate on the film morphology, a diluted L-PLLA/DM solution was cast and dried relatively rapidly. Drug nuclei appear on the solution surface, but most of the DM crystallization occurred in a gel-like concentrated solution. As a result, a structure similar to that of the B-type film was created. The kinetics of solution drying play a major role in the DM mode of dispersion in a polymer film. The opposite process of starting with a concentrated solution and obtaining an A-type morphology did not occur, even when the drying rate was extremely slow. The relatively slow diffusion rate of DM particles in a viscous medium appears to hinder drug crystal formation on the surface of a growing polymer film.

The net effect of DM re-crystallization on morphology was investigated using dilute and concentrated solution conditions. The structures obtained were observed by SEM. The resulting DM powder yielded particles of 0.1-3 μm, partially aggregated. Re-crystallization of DM from dilute solution leads to formation of large rectangular crystals. These crystals are composed of well-packed, small primary particles, similar to those of the resultant powder. These primary particles tended to merge into large, well-arranged structures, due to the slow drying process. The rectangular shapes are most likely a tertiary structure. In contrast, re-crystallization of DM from concentrated over-saturated DM solution, led to formation of a structure similar to the original one. SEM observations showed that the DM distribution within a PLLA film is determined mainly by the kinetics of drug re-crystallization, whereas the polymer chain structure and morphology have a minor effect on the DM distribution. The latter has a significant effect on the film properties.

LM observations of the films enable the observation of film structure. However, a better view of DM dispersion within the B-type film and its particles size could not be observed via LM. Therefore, the morphology of these films was also studied by electron microscopy. TEM and HRSEM micrographs of H-PLLA/DM(B) film revealed DM to be in the form of small rectangular shapes (1-5 μm) in addition to small particles (less than 1 μm), within the whole cross-section area of the film. The micrographs indicated the partial formation of the DM tertiary structure, in spite of the relatively fast drying. The features of the spherulitic PLLA could not be observed, due to their low contrast.

HRSEM micrographs of the cryogenically fractured surface of the film indicated a poor PLLA-DM interphase adhesion. The chemical nature of the DM is aromatic while that of the PLLA is aliphatic. Therefore, the PLLA does not tend to “wet” DM, resulting in poor polymer/drug interphase. The PLLA contains ester groups and each DM molecule contains two carbonyl and three hydroxyl groups. Therefore, specific strong interactions, namely hydrogen bonds, may be formed between the carbonyl oxygens in the PLLA chains and the hydroxyl hydrogens in the DM. However, in this system, these strong intermolecular interactions are created between adjacent DM molecules and particles, leading to strong DM segregation during film drying.

Both kinetic parameters of film formation process and thermodynamic parameters of the system's components affect the film morphology. For example, the rate of solvent evaporation and the resulting rate of drug and polymer crystallization have a significant effect on the drug distribution and its structure. Solubility effects of the system components determine the nature of the starting solution and therefore affect the diffusion processes during drying. Interestingly, PLLA/hydrocortisone and PLLA/curcumin films, prepared from dilute and concentrated solutions, also showed the A-and B-type structures, respectively. These results indicate that the methods of structuring demonstrated above, are general.

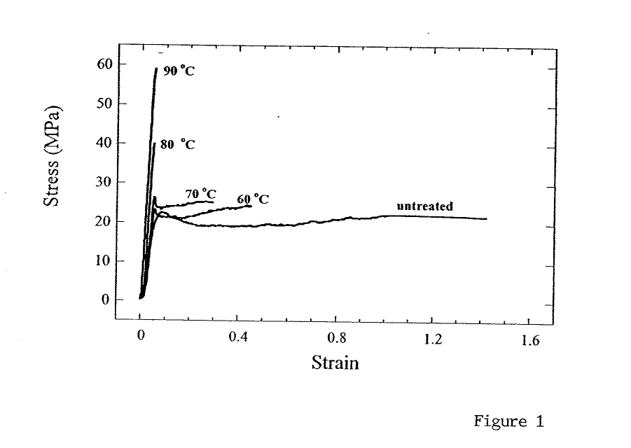

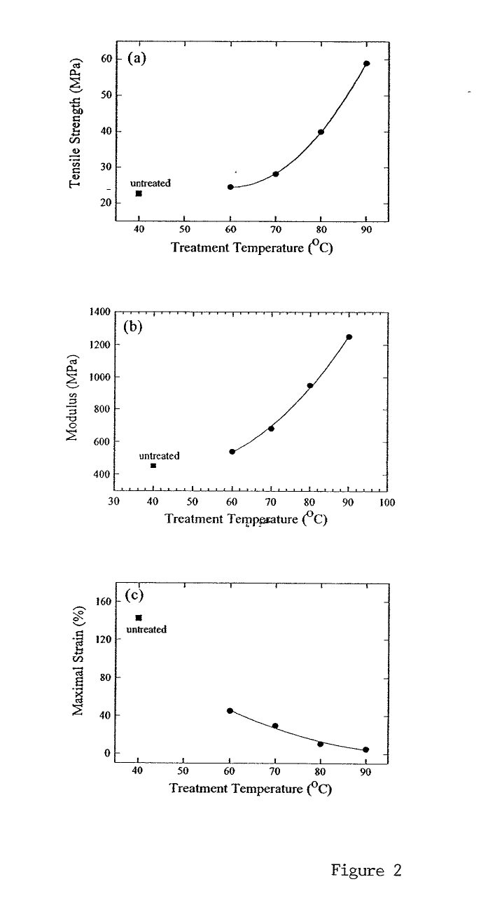

Mechanical Properties in Tension of PLLA/DM Films. The tensile mechanical properties of H-PLLA and L-PLLA films containing DM were compared to those of the neat PLLA films. Stress-strain curves of untreated H-PLLA and H-PLLA treated at various temperatures are presented in FIG. 1. The tensile strength, elastic modulus and maximal strain, as a function of heat treatment temperature are presented in FIG. 2. The untreated, as-cast H-PLLA film was relatively ductile (εₘₐₓ=142%) and had a relatively low tensile strength (σₘₐₓ=22.6 Mpa) and modulus (E=453 MPa). Although a post-preparation isothermal heat treatment did not change the basic film morphology, it affected significantly its mechanical properties. The film became brittle; its strength and modulus increased with the treatment temperature, attaining 59.0 MPa and 1250 MPa, respectively, for the films treated at 90° C. The maximal strain decreased by increasing the treatment temperature, attaining 5.4% for the treated at 90° C. film. It should be noted that the maximal tensile strengths of the ductile films were their yield strengths and those of the brittle ones were just below but very close to, their yield strengths. Hence, the yield strength vs. treatment temperature curve (not shown) was very similar to the tensile strength vs. treatment temperature curve [FIG. 2(a)]. Similar mechanical properties in tension are reported in other studies of PLLA.

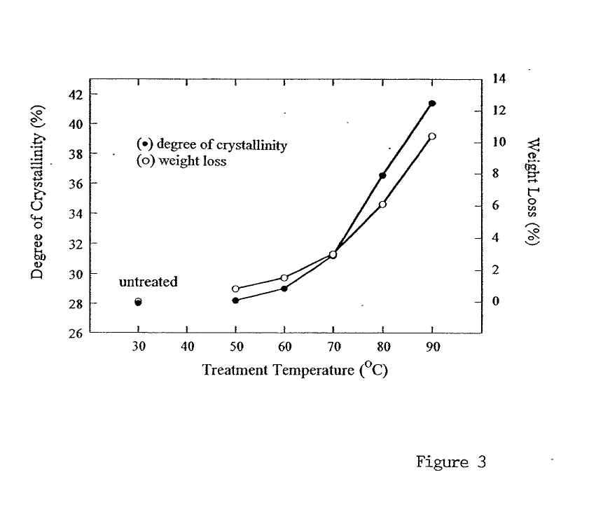

The degree of crystallinity and the percent weight loss of the film due to solvent residues evaporation, were measured and their values, as a function of the heat treatment temperature, are presented in FIG. 3. The degree of crystallinity of the untreated H-PLLA (28.0%) was slightly increased by heat treatment at temperatures below the Tg of the polymer (74° C.). In contrast, a high increase in the degree of crystallinity was obtained by heat treatments at temperatures above the Tg, reaching 41.4% at 90° C. Heat treatment at T>Tg enables motion of segments in the polymer chains and therefore, further crystallization to occur. It is known that chain scission during heat treatment cannot be avoided. The chain scission phenomenon resulted in shorter chains and contributed to the additional crystallization. The additional crystalline material was due mainly to further crystallization of domains between adjacent lamellae and defect corrections. Therefore, a change in the film morphology, due to the high increase in the degree of crystallinity obtained at 90° C., was not expected, nor was it observed. The shape of the weight loss curve in FIG. 3 is similar to the degree of crystallinity curve, i.e., significant weight loss occurs only in films treated above Tg, reaching 10.4% at 90° C. At these relatively high temperatures, solvent molecules that are “trapped” within the film are easily evaporated.

From the results above, both the increase in degree of crystallinity and solvent residues release resulted from the post-preparation heat treatment and affect the mechanical properties of the PLLA film. Well-packed crystals contribute to stiffness and strength. Therefore, a higher degree of crystallinity was effective in increasing the modulus and the yield point. The amorphous phase of a polymer enabled large deformations. Hence, a decrease in the amorphous phase content contributed to the decrease in maximal strain [FIG. 2(c)]. In this system, the solvent residues act as a plasticizer that reduced stiffness, strength and brittleness, since interchain forces were effectively reduced. Thus, untreated PLLA was flexible at temperatures below the Tg, while heat treatment at T>Tg with enhanced release of solvent residues leads to brittle and tough films, as expected of intermediate degree of crystallinity (20-60%) below the Tg, where only small motions of small groups occurs. The exponential shape of the tensile strength and modulus vs. treatment temperature curves [FIGS. 2(a,b)] were similar to those for the degree of crystallinity and weight loss vs. treatment temperature curves [FIG. 3].

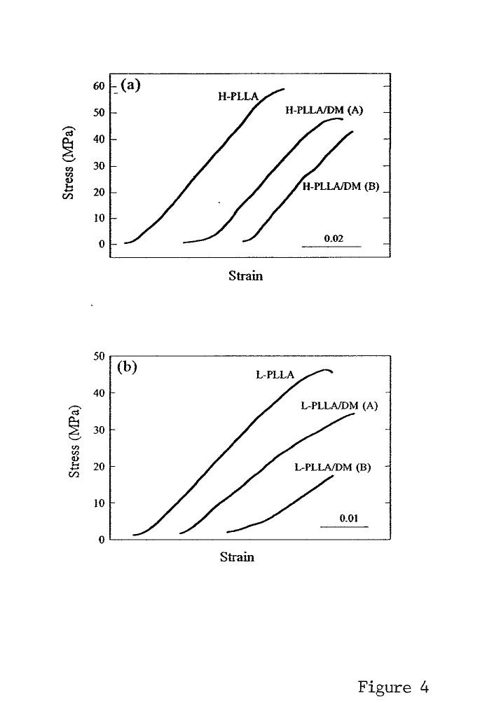

Stress-strain curves of H-PLLA and L-PLLA based films are presented in FIGS. 4(a) and (b), respectively. The measured maximal tensile strength (at break), elastic modulus and maximal strain (at break) are presented in Table 2.

TABLE 2
The Mechanical Properties in Tension of PLLA Based Films.

| | Tensile | | |
|---|---|---|---|
| Film | Strength (MPa) | Modulus (MPa) | Strain (%) |
| H-PLLA (untreated) | 22.6 ± 2.1 | 453 ± 24 | 142 ± 7.8 |
| H-PLLA | 59.0 ± 3.0 | 1250 ± 37 | 5.4 ± 0.40 |
| H-PLLA/DM (A) | 48.0 ± 3.1 | 1182 ± 41 | 5.4 ± 0.55 |
| H-PLLA/DM (B) | 42.7 ± 4.2 | 1165 ± 44 | 3.7 ± 0.41 |
| L-PLLA (untreated) | 19.0 ± 2.3 | 255 ± 15 | 107 ± 7.7 |
| L-PLLA | 46.4 ± 3.8 | 1222 ± 57 | 4.2 ± 0.50 |
| L-PLLA/DM (A) | 34.3 ± 3.1 | 936 ± 56 | 3.7 ± 0.41 |
| L-PLLA/DM (B) | 17.3 ± 2.8 | 870 ± 67 | 2.6 ± 0.40 |

The heat-treated films were relatively brittle and their tensile failure occurs at or slightly beyond the yield stress. The mechanical properties of a polymer/drug film were determined by the film composition, the polymer chain structure and morphology, and the drug distribution. The neat H-PLLA was stronger and tougher than L-PLLA due to the higher molecular weight. The L-PLLA treated film exhibited 53.6% crystallinity, while the H-PLLA treated film was less crystalline (41.4%). Hence, the crystallinity effect was not strong enough to compensate for the molecular weight effect. As a result, the more crystalline L-PLLA was weaker than the less crystalline H-PLLA. In addition to their better strength and stiffness, the H-PLLA films were more ductile than the L-PLLA. Raising the molecular weight, which increased brittle strength and reduced degree of crystallinity, decreased the chance of brittle failure. Also, polymers with smaller, finer-textured spherulites tended to fail at relatively high strains, while those with large, coarse spherulites often failed by brittle fracture between spherulites at low strains. The L-PLLA spherulites (50-100 μm diameter) were larger than those of the H-PLLA (less than 10 μm). Hence, the better ductility of the H-PLLA was obtained due to differences in molecular weight, percent crystallinity and the polymer texture.

In general, incorporation of DM in H-PLLA or L-PLLA decreased the strength, modulus and maximal strain (FIG. 4 and Table 2). As previously mentioned, the treated PLLA is relatively brittle and its tensile failure occurs at or slightly beyond the yield stress. The change in ductility due to addition of the drug was not significant. However, in the B-type films, failure occured below the yield point. In the A-type films, the DM was located on the surface of the PLLA matrix, the polymer structure was very similar to that of the neat PLLA and therefore, the changes in mechanical properties were relatively small.

In contrast, in the B-type films, the DM was located within the PLLA. As a result, for both H-PLLA and L-PLLA, the deterioration of mechanical properties due to DM incorporation was more significant than in the A-type film. The DM molecules have a 4 ring planar structure and therefore, function as stiff organic fillers, increasing stiffness and strength. However, the totally different nature of these components, and the lack of interactions between them, contributed to poor interphase adhesion and resulted in deterioration of mechanical properties. The deterioration of mechanical properties due to drug incorporation was more significant for the L-PLLA film than for the H-PLLA film, especially in the B type films. The relatively large amount of amorphous phase and fine spherulitic structure of H-PLLA can better tolerate drug incorporation than the smaller amount of amorphous phase and coarser structure of L-PLLA.

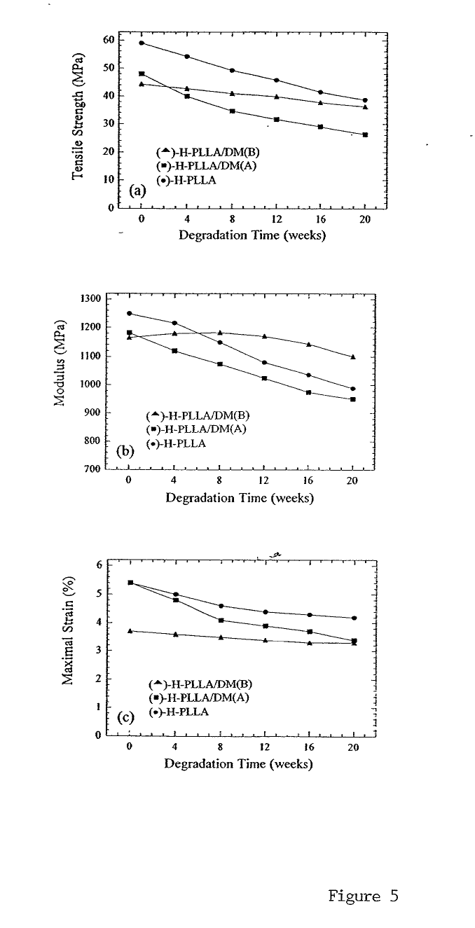

In Vitro Study of PLLA/DM Films. The PLLA/DM films were floated on sterile water at 37° C. in order to investigate degradation of mechanical properties in vitro. The mechanical properties in tension of H-PLLA based films as a function of time are presented in FIG. 5. In general, the tensile strength, modulus and ductility decreased with the increase in floatation time. All three types of films, H-PLLA, H-PLLA/DM(A) and H-PLLA/DM(B), maintained relatively good mechanical properties throughout the twenty week period of study.

The tensile strength of neat H-PLLA and H-PLLA/DM(A) films exhibited parallel linear decreases with time [FIG. 5(a)]. The strength of the A type film was consistently lower than that of the neat H-PLLA film, due to the low mechanical properties of the layers near the polymer/DM interphase. Interestingly, the tensile strength of the B-type film also decreased linearly with time, but the slope was smaller than those of neat H-PLLA and A-type films. Thus, the DM incorporation in the film contributed to better retention of film strength upon exposed to water.

Similarly, the Young's modulus of H-PLLA/DM(A) film was lower than that of the neat H-PLLA film throughout the twenty weeks of study [FIG. 5(b)]. The H-PLLA/DM(B) film's modulus did not change appreciably during the first twelve weeks study and was slightly reduced afterwards. These films were relatively brittle at their onset points and exhibited only several percents of strain, due to the post-casting heat treatment (see the previous section). The H-PLLA and H-PLLA/DM(A) films were more ductile than H-PLLA/DM(B) throughout the twenty weeks of study [FIG. 5(c)]. However, ductility of H-PLLA/DM(B) showed almost no change with time. The decrease in maximal strain was equivalent to the decrease in tensile strength and modulus for the studied H-PLLA-based films.

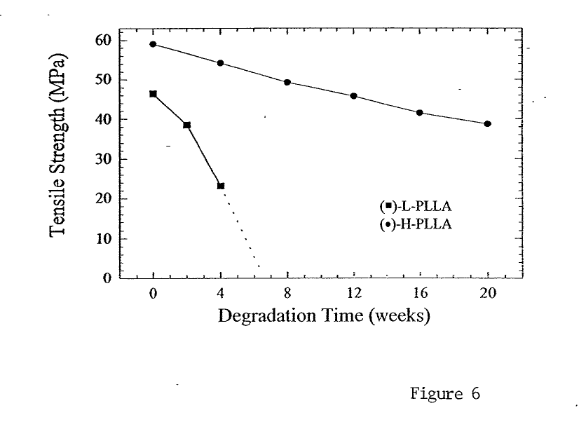

A different mechanical property behavior was observed for L-PLLA films. The deterioration in their mechanical properties was much faster than that observed for those based on H-PLLA, as demonstrated in FIG. 6. While the H-PLLA film lost less than 10% of its initial tensile strength after 4 weeks flotation on sterile water, the L-PLLA film showed a much greater decrease in tensile strength of approximately 50%. L-PLLA films became very brittle and could not be handled after longer periods of floatation time.

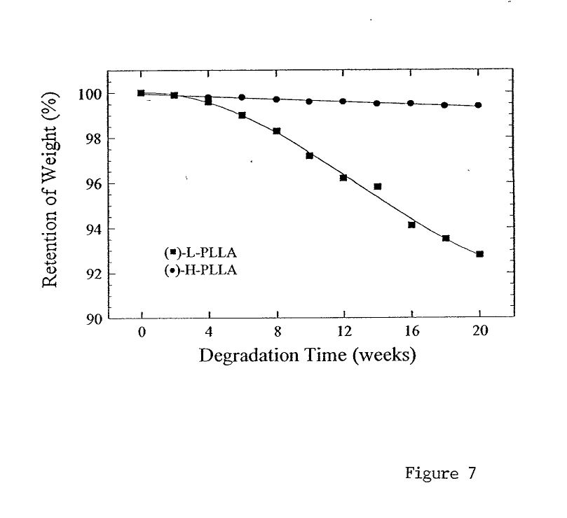

The films' weight retention and morphology were studied to elucidate and understand the mechanical property behavior. While the H-PLLA film retained its initial mass and exhibited a decrease of less than 1% in weight after 20 weeks of degradation, the L-PLLA started losing weight after 2 weeks and exhibited a decrease of approximately 8% after 20 weeks (FIG. 7).

SEM was used to compare the morphology of cryogenically fractured surfaces of H-PLLA and L-PLLA films after exposure to water for 20 weeks with the morphology of the unexposed films. The characteristic features of the H-PLLA film after exposure to water for twenty weeks remained similar to those of the original one, but the topography became rougher. In contrast, significant changes in morphology were observed for the L-PLLA film after exposure to water. Two characteristic textures were observed: a rough fractured surface, typical of an eroded polymer, and a fine microporous structure (0.2-1 μm in diameter). The former texture is more typical of the film surface while the latter is more typical of the interior. These observations are in agreement with those in the literature in that degradation does not occur homogeneously, but rather more quickly in the interior than on the surface, due to acidic self-catalysis. In addition to these changes in microstructure, the L-PLLA-based films also developed visual changes. After 4 weeks of exposure to water, white spots appeared and grew with time, until the films had a marble-like appearance. These white spots are due to the presence of crystalline agglomerates. In contrast, the H-PLLA based samples did not show any visual changes.

Bioresorbable polymers undergo five general stages of degradation:

(a) Hydration—absorption of water from the surrounding.

(b) Depolymerization or chemical cleavage of the polymer backbone. This results in reduction in the average molecular weight and mechanical properties. The mass remains unchanged.

(c) Loss of mass, which occurs when the polymer begins to fragment into pieces of low molecular weight. Progressive degradation changes the microstructure of the polymer through the formation of pores, via which oligomers and monomers are released, leading to the weight loss of the polymer.

(d) Absorption—assimilation of small fragments by phagocytes and dissolution of monomeric anions in the fluid.

(e) Elimination—through Krebs cycle metabolism.

When the polymer is not exposed to living cells, medium stages (d) and (e) do not occur.

Another approach distinguishes simply between degradation and erosion. Accordingly, the process of “degradation” describes the chain scission process, during which polymer chains are cleaved to form oligomers and finally, to form monomers. “Erosion” designates the loss of material owing to monomers and oligomers leaving the polymer. The degradation and morphology studies (FIG. 7) demonstrated that during the 20 week study the H-PLLA films undergo degradation (stages a and b). Their mass remained practically unchanged and the relatively small deterioration in tensile mechanical properties can be attributed to: (1) chain scission, leading to decrease in the average molecular weight, and (2) morphological changes. In addition to degradation (stages a and b), the L-PLLA films undergo erosion (stage c), starting after two weeks of exposure to aqueous medium. These structural changes enable release of low molecular weight fragments.

The deterioration of tensile mechanical properties was faster for L-PLLA films than for H-PLLA ones, due to the following reasons:

(a) The initial molecular weight of L-PLLA (i.v.=1.02 dL/g) is lower than that of H-PLLA (i.v.=3.6 dL/g). Hence, low molecular weight fragments are obtained after a relatively short period of time, giving rise to erosion and pore formation. Film samples have relatively high surface/volume ratio and therefore, their rate of degradation is relatively fast.

(b) PLLA is degraded by simple hydrolysis. The hydrolysis starts in the amorphous phase of the polymer, due to relatively easy penetration of water to these domains. The degree of crystallinity and spherulite size of the L-PLLA are higher than those of the H-PLLA polymer. While the former exhibits 53.6% crystallinity and large spherulites of 50-100 μm, the latter exhibits 41.4% crystallinity and relatively small spherulites (less than 10 μm). Hence, The L-PLLA contains smaller portion of crystalline phase, located mainly around the spherulites and between adjacent lamellae, in a relatively low volume. The amorphous phase of L-PLLA undergoes more massive degradation than that of H-PLLA, leading to its faster destruction. The failure of the film in tension probably starts in an amorphous domain.

In Vitro Study of Expandable PLLA/DM Stents. A novel expandable tubular stent was developed from these films. Holes of 3 mm diameter may be made in the film to enable contaminant removal from the airway in the stent vicinity.

The initial radial compression properties of H-PLLA and L-PLLA stents formed in this configuration are presented in Table 3.

TABLE 3
Radial Compression Properties of PLLA based Stents.

| Film type | Strength (KPa) | Initial deformation pressure (KPa) |
|---|---|---|
| H-PLLA | >200 | >200 |
| H-PLLA/DM(A) | >200 | >200 |
| H-PLLA/DM(B) | >200 | >200 |
| L-PLLA | 158 ± 23 | 138 ± 13 |
| L-PLLA/DM(A) | 145 ± 17 | 131 ± 12 |

The maximal applied pressure using a radial compression chamber was 200 KPa, therefore higher strength values could not be measured. In general, the H-PLLA based stents were stronger than the L-PLLA stents. The H-PLLA stents could endure a radial compression pressure of at least 200 KPa without exhibiting any deformation. The L-PLLA stents started to deform at 138 KPa and broke at 158 KPa. The L-PLLA stents exhibited a small elastic deformation before brittle failure. Incorporation of DM in the film reduced the stent's radial compression strength, and it started to deform at lower pressure. However, both stent types, H-PLLA and L-PLLA, demonstrated initial radial compression strengths at least 50 times higher than the strength required for the tracheal application (approximately 3 KPa). The tensile mechanical properties of a polymer are more sensitive to poor polymer-filler interphase adhesion than compression properties. Therefore the stent's compression strength (Table 3) was less sensitive to drug incorporation than the tensile mechanical properties of the film (Table 2).

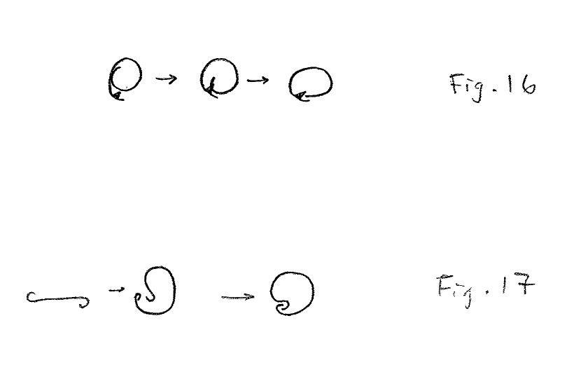

A locking mechanism may be incorporated into the lumen or the exterior of the film that locks the stent, once opened or unfolded, into place (FIGS. 16 and 17). The locking structure maybe, e.g, that extends at least partially along the length of the film. The lock may be along the entire length of the film or at specific locations. It is expected that the lock will prevent the film from “re-rolling” under pressure from the vessel where it is inserted. It is also expected that the lock may provide for the lumen to be about equivalent along the length of the stent, or even varied along the stent where the vessel to be kept open does not have a lumen of a consistent diameter. Further, the lock could be located on the inside or the outside of the stent. If located on the outside, the stent can be opened and released.

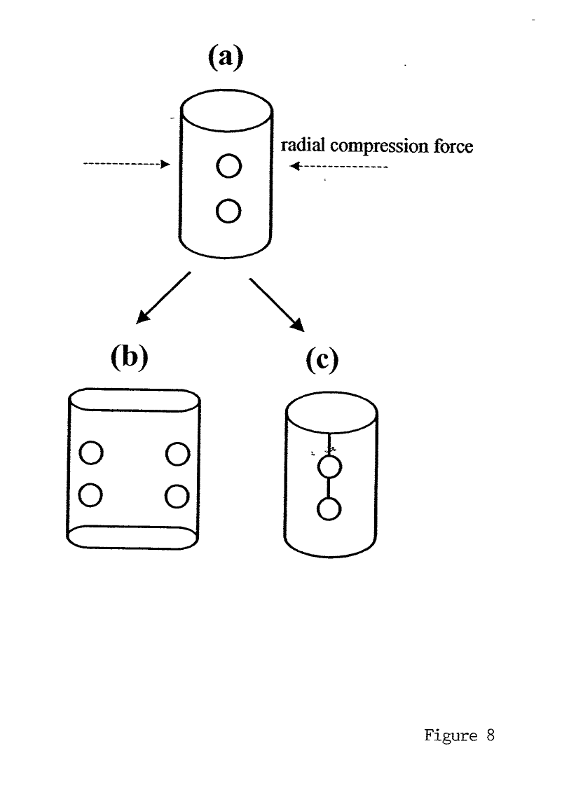

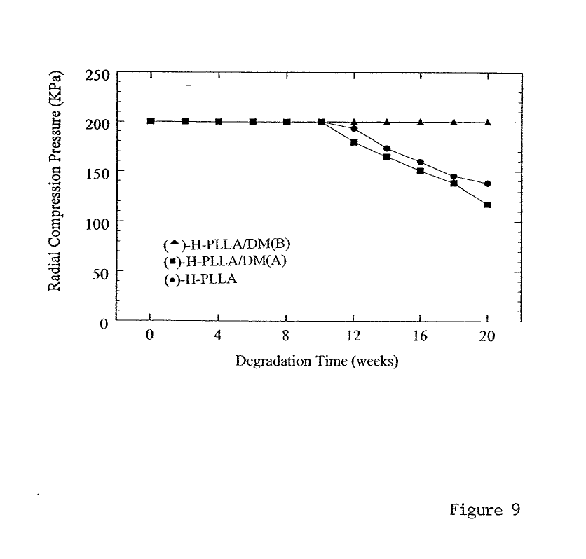

The stents were immersed in sterile water at 37° C. in order to investigate the effect of their in vitro degradation on mechanical properties. Two modes of failure were obtained, as presented in FIG. 8. The H-PLLA stents tended to deform in the radial direction and exhibit an elliptical shape. The deformation increased with radial compression pressure, until the stent was almost flattened, as shown in FIG. 8(b). The stent returned to its original tubular shape after removing the radial pressure. Hence, the exhibited deformation was elastic. The deformation failure was obtained only for samples that were immersed for more than 10 weeks.

In contrast, the L-PLLA stents, prepared from a weaker and more brittle polymer, exhibited a small deformation in the radial direction and then developed a fracture in the longitudinal direction, between two adjacent holes, as shown in FIG. 8(c). The stents that were immersed for more than 2 weeks exhibit the described fracture without showing any earlier deformation. Hence, the radial pressure needed in order to start a deformation was considered as a failure point for the H-PLLA stents and the radial pressure needed to create a fracture was considered as a failure point for the L-PLLA stents. The latter failure is not desired, since it appears suddenly, without showing any prior signs. In both cases, the deformation or defect started at a relatively weak point, i.e., a local defect in structure or a relatively small local degree of crystallinity.

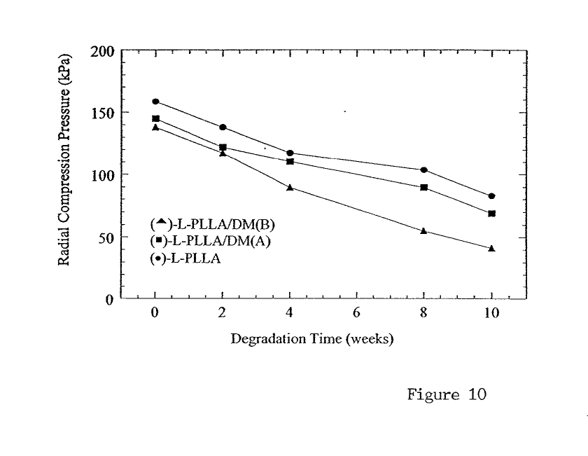

The radial pressure needed in order to start an elastic (reversible) deformation in the radial direction for the H-PLLA stents, as a function of immersion time, is presented in FIG. 10. The H-PLLA/DM(B) stent did not show any elastic deformation, after applying 200 KPa, throughout the 20 weeks studied. The radial compression pressure needed to initiate deformation in the neat H-PLLA and H-PLLA/DM(A) stents, was also not lower than 200 KPa for the first 10 weeks and then showed a linear decrease with time. In general, the H-PLLA stents demonstrated good strength and subsequent ductility. Hence, while becoming weaker, due to further degradation with time, the H-PLLA stents will not exhibit an unexpected brittle fracture for a relatively long period of time. Dexamethasone contributed to better radial compression strength, while being located within the H-PLLA film, due to its inherent stiffness.

The radial compression pressure needed to create a fracture in the L-PLLA stents is presented as a function of immersion time in FIG. 10. An almost linear decrease in breaking pressure with an increase in immersion time was obtained for all 3 stent types. The breaking pressures of the L-PLLA stents were higher than those of the L-PLLA stents containing DM. Stents prepared from the A-type film have better resistance than those prepared from the B-type film. The radial compression strength of the L-PLLA stents could be measured only for stents that were immersed less than 10 weeks. Longer immersion times resulted in very brittle stents that could not be handled.

The mechanical properties of H-PLLA based stents were better than those of L-PLLA ones. They combined high strength that could be retained for relatively long period of time, with good flexibility. Incorporation of the steroidal drug DM on the surface of the H-PLLA film (A-type) did not affect the stent's mechanical properties. Incorporation of DM in the film (B-type) resulted in better mechanical properties. Therefore, H-PLLA stents are more applicable for supporting the trachea. In contrast, L-PLLA stents can support the trachea for approximately two months. Although the radial compression pressure that they can endure is higher than the requested value, their brittleness is not desired. Incorporation of DM in the film reduces the mechanical properties of the L-PLLA stents.

Optical methods for both external imaging and for incorporation into medical devices have been developed. Devices for imaging or measuring spectroscopic features of living tissue include U.S. Pat. No. 5,137,355, U.S. Pat. No. 5,203,339, U.S. Pat. No. 5,697,373, U.S. Pat. No. 5,722,407, U.S. Pat. No. 5,782,770, U.S. Pat. No. 5,865,754, WO 98/10698. Optical contrast agents, such as indocyanine green, coupling of these systems to medical instruments using exogenous contrast to locate or target specific tissue sites may be used in conjunction with the polymer film stent of the present invention to provide non-invasive detection of the status of the stent.

Invasive or contact-marking medical instruments equipped with optics, such as catheters, needles, and trocars, have been described in, e.g., U.S. Pat. No. 5,280,788, U.S. Pat. No. 5,303,026, U.S. Pat. No. 5,349,954, U.S. Pat. No. 5,413,108, U.S. Pat. No. 5,596,992, U.S. Pat. No. 5,601,087, U.S. Pat. No. 5,647,368, U.S. Pat. No. 5,800,350, relevant portions incorporated herein by reference. Some of these devices are endoscopic, and do not image through opaque tissue. Others are invasive medical instruments and devices, but they do not detect or localize target tissues using exogenous contrast, nor do they allow targeting of an instrument to specific tissue sites using an in vivo contrast signal from an exogenous contrast agent.

An example of a contrast agent may include an emitting reporter. In this regard, use of emitting reporters is known in the art. For example, in nuclear medicine and PET scanning, agents that spontaneously emit a particle or photon provide a signal to identify to localization of the emitter agent. Targeted instruments based upon non-optical emitters have been built (e.g., U.S. Pat. No. 5,857,463). A drawback to nearly all emitter-based systems is that they suffer from low signal, which is due to use of radioactive or ionizing emitters that produce a signal only intermittently (such as a particle decay) and at low intensity, forcing long integration times that make real time imaging and precise localization slow or difficult. This low signal presents a particular difficulty when using a moving medical instrument, or when targeting a tissue using a moving probe, both of which require a strong signal for reliable, rapidly updated real time analysis.

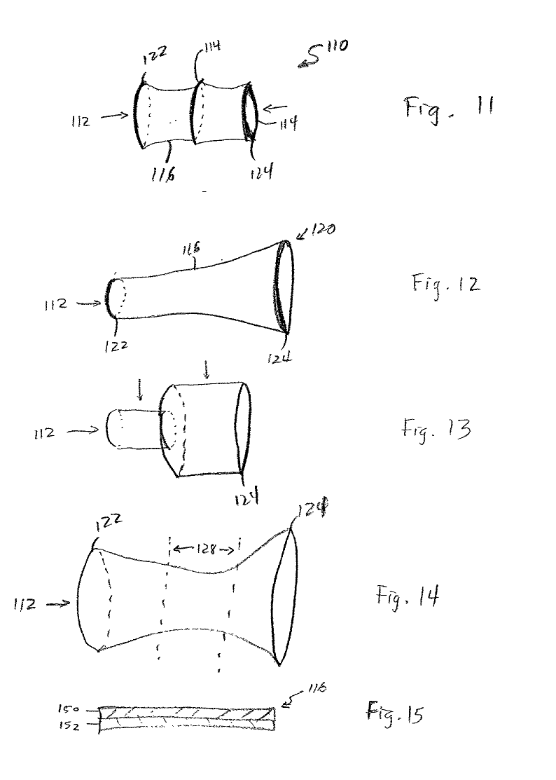

Various forms of the stent are possible. FIG. 11 depicts a stent having two structural rings around the diameter of the cylinder. The structural rings may be expandable to conform to the cylinder and could be made from a variety of materials including metals and plastics. The support rings could be spaced apart, and may be removable. The rings will provide additional support for the stent, a feature that would be important during the growth of the vessel or airway and perhaps, during pressure changes within a vessel. Additional forms of the stent include cylinders having two ends of different diameter that open into the lumen of the stent (FIG. 12), or alternatively, having two ends of equal diameter and a variable middle section (FIG. 14). Another form of the stent may have two cylinders of different size positioned such that the smaller one fits into the larger one (FIG. 13). These designs are advantageous for use in vessels that have variable thickness.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A structural support stent for use in a body lumen comprising:
a structural self-expanding biocompatible material that forms a cylinder having an inner surface and an outer surface.

2. The structural support stent recited in claim 1, wherein the biocompatible material further comprises a bioresorbable material.

3. The structural support stent recited in claim 1, wherein the biocompatible material is chosen from the group consisting of poly DL-lactic acid, poly L-lactic acid, polyglycolic acid, polycaprolactone, polydioxanone, or mixtures thereof.

4. The structural support stent recited in claim 1, further comprising a shape memory that allows the stent to self-expand to a predetermined size.

5. The structural support stent recited in claim 1, further comprising a locking mechanism on the outside of the stent that extends along the location of the film.

6. The structural support stent recited in claim 1, further comprising a locking mechanism positioned at particular points along the outside of the stent.

7. The structural support stent recited in claim 1, further comprising a locking mechanism on the inside of the stent that extends along the location of the film.

8. The structural support stent recited in claim 1, further comprising a locking mechanism positioned at particular points along the inside of the stent.

9. The structural support stent recited in claim 1, further comprising a cylinder of variable width.

10. The structural support stent recited in claim 1, further comprising a cylinder with two ends of equal diameter and a middle section with a different diameter than the ends.

11. The structural support stent recited in claim 1 comprising a cylinder having a first and second end wherein the diameter of the first end is greater than the diameter of the second end.

12. The structural support stent recited in claim 1 comprising two cylinders that differ in diameter wherein one cylinder can be positioned inside of the other.

13. The structural support stent recited in claim 1, further comprising perforations that create openings from the inner surface to the outer surface.

14. The structural support stent recited in claim 1, further comprising expandable rings around the support that provide strength.

15. The structural support stent recited in claim 1, further comprising one or more drugs contained in the biocompatible material.

16. The structural support stent recited in claim 6, wherein the drugs are chosen from the group consisting of steroids, anti-inflammatory formulations and antineoplastics.

17. The structural support stent recited in claim 6, wherein the drug comprises dexamethasone.

18. The structural support stent recited in claim 4, wherein the concentration of the drug varies from the outer surface to the inner surface.

19. The structural support stent recited in claim 1, where the biocompatible material comprises a laminate of two or more layers.

20. The structural support stent recited in claim 10, further comprising one or more drugs contained in at least one of the layers.

21. The structural support stent recited in claim 1, further comprising a substance on the surface of the stent that allows for visualization of deformation.

22. The structural support stent recited in claim 21, wherein the substance on the surface of the stent further comprises a contrast agent.

23. A method of preparing a self-expanding support stent having an inner surface and an outer surface comprising the steps of:
forming a film of a biocompatible material;
rolling the film onto a mandrel;
annealing the film while on the mandrel; and
removing the cured film from the mandrel.

24. The method recited in claim 12, wherein the biocompatible material further comprises a bioresorbable material.

25. The method recited in claim 12, wherein the biocompatible material is chosen from the group comprising poly DL-lactic acid, poly L-lactic acid, polyglycolic acid, polycaprolactone, polydioxanone, or mixtures thereof.

26. The method recited in claim 12, wherein the forming further comprises film casting, melt processing, injection molding, or film blowing.

27. The method recited in claim 12, wherein the mandrel has a diameter that is a desired inner diameter for the structural support stent.

28. The method recited in claim 12, wherein the film is a laminate of one or more layers, each layer comprising a different biocompatible material.

29. The method recited in claim 12, further comprising the step of rolling one or more additional films of biocompatible material onto the mandrel to form a laminate.

30. The method recited in claim 12, wherein the film further comprises one or more drugs.

31. The method recited in claim 19, wherein the drugs are chosen from the group comprising: steroids, anti-inflammatory formulations, and antineoplastics.

32. The method recited in claim 19, wherein the drug comprises dexamethasone.

33. The method recited in claim 18, wherein each film in the laminate further comprises one or more drugs.

34. The method recited in claim 22, wherein the drugs are chosen from the group comprising: steroids, anti-inflammatory formulations and antineoplastics.

35. The method recited in claim 22, wherein the drug comprises dexamethasone.

36. The method recited in claim 12, wherein the annealing is performed in a vacuum.

37. The method recited in claim 12, wherein the annealing further comprises heating the biocompatible material and the mandrel.

38. The method recited in claim 12, wherein the annealing comprises heating the biocompatible material and mandrel to a temperature above the glass transition of the biocompatible material.

39. The method recited in claim 12, wherein the annealing comprises heating, in a vacuum, the biocompatible material and the mandrel to a temperature above the glass transition temperature of the biocompatible material.

40. The method recited in claim 12, further comprising the step of perforating the film so that the perforations create openings between the outer surface and the inner surface when the stent has self-expanded.

41. A method of preparing a self-expanding support stent comprising the steps of:
preparing a solution containing a biocompatible material and one or more drugs;
casting a film of the biocompatible material and drugs;
rolling the film onto a mandrel; and
annealing the film while on the mandrel.

42. The method recited in claim 30, wherein the biocompatible material further comprises a bioresorbable material.

43. The method recited in claim 29, wherein the biocompatible material is chosen from the group comprising poly DL-lactic acid, poly L-lactic acid, polyglycolic acid, polycaprolactone, polydioxanone, or mixtures thereof.

44. The method recited in claim 30, wherein the drugs are chosen from the group comprising: steroids, anti-inflammatory formulations and antineoplastics.

45. The method recited in claim 30, wherein the drug comprises dexamethasone.

46. The method recited in claim 30, wherein the mandrel has a diameter that is a desired inner diameter for the structural support stent.

47. The method recited in claim 30, wherein the film is a laminate of one or more layers, each layer comprising a different biocompatible material.

48. The method recited in claim 30, further comprising the step of rolling one or more additional films of biocompatible material onto the mandrel to form a laminate.

49. The method recited in claim 30, wherein the curing is performed in a vacuum.

50. The method recited in claim 30, wherein the curing further comprises heating the biocompatible material and the mandrel.

51. The method recited in claim 30, wherein the annealing comprises heating the biocompatible material and mandrel to a temperature above the glass transition of the biocompatible material.

52. The method recited in claim 30, wherein the annealing comprises heating, in a vacuum, the biocompatible material and the mandrel to a temperature above the glass transition temperature of the biocompatible material.

53. The method recited in claim 30, further comprising the step of perforating the film so that the perforations create openings between the outer surface and the inner surface when the stent has self-expanded.

54. A method for deploying a self-expanding structural support stent comprising the steps of:
reducing the diameter of the stent by coiling;
stabilizing the coiled stent in the coiled position;
placing the stent in a body lumen; and
releasing the stabilization of the coiled stent to allow self-expansion.

55. The method recited in claim 43, wherein the stabilization comprises clamping the coiled stent.

56. The method recited in claim 43, wherein the stabilization comprises placing the coiled stent in a sleeve.

57. The method recited in claim 43, wherein the placement of the coiled stent within the body lumen comprises the use of a laryngoscope.

58. The method recited in claim 43 wherein the placement of the coiled stent within the body lumen is accomplished under direct visualization.

59. The method recited in claim 43, wherein the body lumen comprises the trachea

60. A method for incorporating a drug within a biocompatible film comprising the steps of:
dissolving a biocompatible material and suspending a finely divided drug in a solvent;
solution casting and relatively fast solvent drying;
rolling the film onto a mandrel;
annealing the film while on the mandrel; and
removing the annealed film from the mandrel.

61. A method for incorporating a drug on the surface of a biocompatible film comprising the steps of:
dissolving a biocompatible material and a drug in a solvent;
solution casting and relatively slow solvent drying;
rolling the film onto a mandrel;
annealing the film while on the mandrel; and
removing the annealed film from the mandrel.

62. A method for creating a gradient of a drug across a biocompatible film comprising the steps of:
preparing several biocompatible films with different drug contents;
binding these films together to create a multi-layer film;
rolling the multi-layer film onto a mandrel;
annealing the multi-layer film while on the mandrel; and
removing the annealed multi-layer film from the mandrel.
